(19) United States
(12) Patent Application Publication
(10) Pub. No.: US 2008/0282764 A1
(43) Pub. Date: Nov. 20, 2008

(54) Calibration checking for continuous emissions monitoring system

(75) Inventors: Mark Holt, Emmaus, PA (US); William Eberhardt, Cherry Hill, NJ (US)

Correspondence Address: GE ENERGY GENERAL ELECTRIC;C/O ERNEST G. CUSICK, ONE RIVER ROAD, BLD. 43, ROOM 225, SCHENECTADY, NY, 12345, US

(73) Assignee: General Electric Company

(21) Appl. No.: 11/803,617

(22) Filed: May 15, 2007

(51) Int. Cl. G01N 33/00; G01N 33/22

(52) U.S. Cl. 73/1.03; 73/23.31

(57) ABSTRACT

A continuous emissions monitoring system is in fluid communication with a flue stack conducting exhaust gas from a combustion source. The continuous emissions monitor system comprises an analyzer for measuring concentrations of an analyte present in the exhaust gas. A probe is in fluid communication with the flue stack to acquire a sample of exhaust gas from the flue stack. The probe is also in fluid communication with and located upstream of the analyzer. The probe tends to remove analyte from the sample. A calibration checking system is in fluid communication with the probe. The calibration checking system includes a source that provides a flow of a known concentration of calibration material to be measured by the analyzer. The calibration material is the same as the analyte. A humidifier is associated with the source to provide moisture to a flow of calibration material. The moisture acts to cleanse removed analyte from the probe and thereby enable an accurate measurement of the concentration of the calibration material.

### BACKGROUND OF THE INVENTION

The present invention relates generally to continuous emissions monitoring of exhaust flue gas streams. More specifically, the present invention relates to calibration checking of continuous emissions monitoring systems.

The United States Environmental Protection Agency (EPA) identifies sources of mercury (Hg) emissions in the U.S. to be utility boilers, waste incinerators that burn mercury-containing wastes (municipal and medical), coal-fired industrial boilers and cement kilns that burn coal-based fuels. A particularly significant source of mercury emissions is coal-fired power plants.

To quantify the emissions from a particular source, a mercury continuous emissions monitoring system (CEMS) is employed. There are three forms of mercury in exhaust flue gas stream of a coal fired power plant that may be monitored by a CEMS. These forms are gaseous elemental mercury, gaseous oxidized mercury and particulate bound mercury that is either elemental or oxidized, at stack gas temperatures in excess of 200° F.

Mercury in the gaseous forms is relatively sticky and has a strong affinity to attach to a wide variety of interior surfaces of CEMS components. Such gaseous mercury is extremely difficult to handle and transport through an extractive gas sampling system to a gas analyzer for measurement. Since exhaust flue gases usually contain relatively low levels of gaseous mercury that must be detected, the small amount of gaseous mercury present that readily attaches to surfaces of the components of the CEMS renders any measurement made on the sample not truly representative of what is conducted in the exhaust stack. Particulates and other undesirable material from the stack gas sample might adhere to the wetted surfaces of the CEMS components and cause the adsorption of elemental mercury onto the wetted surfaces.

The EPA has mandated restrictive controls on mercury emissions. A total mercury measurement is required for regulatory monitoring and the evaluation of mercury control technologies and manufacturing processes requires accurate measurements of gaseous mercury. One example is that the EPA requires a “span gas check” accuracy of plus or minus ten percent (±10%) of a sample range. Accordingly, there exists a need for the development of a reliable and accurate technology capable of verifying the measurement of mercury emitted in an exhaust flue gas stream.

### SUMMARY OF THE INVENTION

One aspect of the present invention is directed to a continuous emissions monitoring system that is in fluid communication with a flue stack conducting exhaust gas from a combustion source. The continuous emissions monitoring system comprises an analyzer for measuring concentrations of an analyte present in the exhaust gas. A probe is in fluid communication with the flue stack to acquire a sample of exhaust gas from the flue stack. The probe is also in fluid communication with and located upstream of the analyzer. The probe tends to remove analyte from the sample. A calibration checking system is in fluid communication with the probe. The calibration checking system includes a source that provides a flow of a known concentration of calibration material to be measured by the analyzer. The calibration material is chemically the same as the analyte. A humidifier is associated with the source to provide moisture to the flow of calibration material. The moisture acts to cleanse removed analyte from the probe and thereby enable an accurate measurement of the concentration of the calibration material.

Another aspect of the present invention is directed to an improved continuous emissions monitoring system that is in fluid communication with a flue stack conducting exhaust gas from a combustion source. The continuous emissions monitor system has an analyzer for measuring concentrations of mercury present in the exhaust gas. A probe is in fluid communication with the flue stack to acquire a sample of exhaust gas from the flue stack and in fluid communication with and located upstream of the analyzer. The probe tends to remove mercury from the sample. A calibration checking system is in fluid communication with the probe. The calibration checking system includes a source that provides a flow of a known concentration of a gaseous species of mercury to be measured by the analyzer. The improvement comprises a humidifier operatively connected with the source to provide moisture to gaseous species of mercury flowing through the humidifier. The moisture acts to cleanse removed mercury from the probe and thereby enable accurate measurement of the concentration of the gaseous species of mercury.

Yet another aspect of the present invention is directed to a method of continuous emissions monitoring of a flue stack conducting exhaust gas from a combustion source. The method comprises the steps of acquiring a sample of exhaust gas from the flue stack with a probe. The probe tends to remove analyte from the sample. Concentrations of the analyte are measured with an analyzer located downstream of the probe. The calibration of the analyzer is checked with a flow of a known concentration of calibration material provided by a source. The calibration material is chemically the same as the analyte. The flow of calibration material is humidified with moisture. The moisture acts to cleanse removed analyte from the probe and thereby enable an accurate measurement of the concentration of the calibration material.

### BRIEF DESCRIPTION OF THE DRAWINGS

FIG. 1 is a schematic illustration, partly in section, of a system, according to one aspect of the invention, for calibration checking of continuous emissions monitoring; and

FIG. 2 is a schematic illustration similar to FIG. 1, of a system, according to another aspect of the invention, for calibration checking of continuous emissions monitoring.

### DETAILED DESCRIPTION OF THE INVENTION

A mercury continuous emissions monitoring system (CEMS) normally consists of a tubular probe assembly located in fluid communication with a flue stack for acquiring a gaseous exhaust sample. The CEMS also includes instrumentation located some distance away from the probe assembly to analyze the acquired sample for the presence of mercury. The amount of mercury present in the exhaust gas stream is continuously measured and recorded. Over time, the total amount of mercury emitted is established. Accuracy and precision of the continuous emissions monitoring system are important.

A critical component of the mercury CEMS is the tubular probe assembly located in fluid communication with the stack for taking the sample. The tubular probe assembly experiences multiple problems. Particulate matter is always present in the in the exhaust stack gas stream and tends to be separated from the exhaust gas and accumulate on surfaces of the tubular probe assembly. Accumulated particulate reduces the accuracy of the mercury measurement. Accumulation of particulates can also result in a reduction of the amount of time the mercury CEMS is accurately measuring emissions in the exhaust gas stream that is mandated by governmental regulation.

The probe assembly is generally U-shaped with an inlet through which samples are drawn and outlet through which samples are discharged. An inertial filter may or may not be located near the probe assembly inlet. A venturi eductor is located near the probe assembly outlet and is supplied by a source of clean heated air that exits from the probe assembly outlet into the exhaust stack gas stream.

This flow of eductor air generates a high velocity (70-100 feet per second) gas flow through the probe assembly, creating a vacuum at the gas inlet. This vacuum at the gas inlet draws the sample stack gas into the probe assembly. Experience has shown that despite the high flow rate, particulate matter does accumulate on surfaces of the probe assembly. This causes inaccuracies of the measurement of mercury in the exhaust gas stream, increasing maintenance and down time.

Since the tubular probe assembly is mounted on the exhaust stack, access to the probe and therefore maintenance of the probe assembly is difficult and time consuming. It is desirable that the probe assembly be as reliable and maintenance-free as possible.

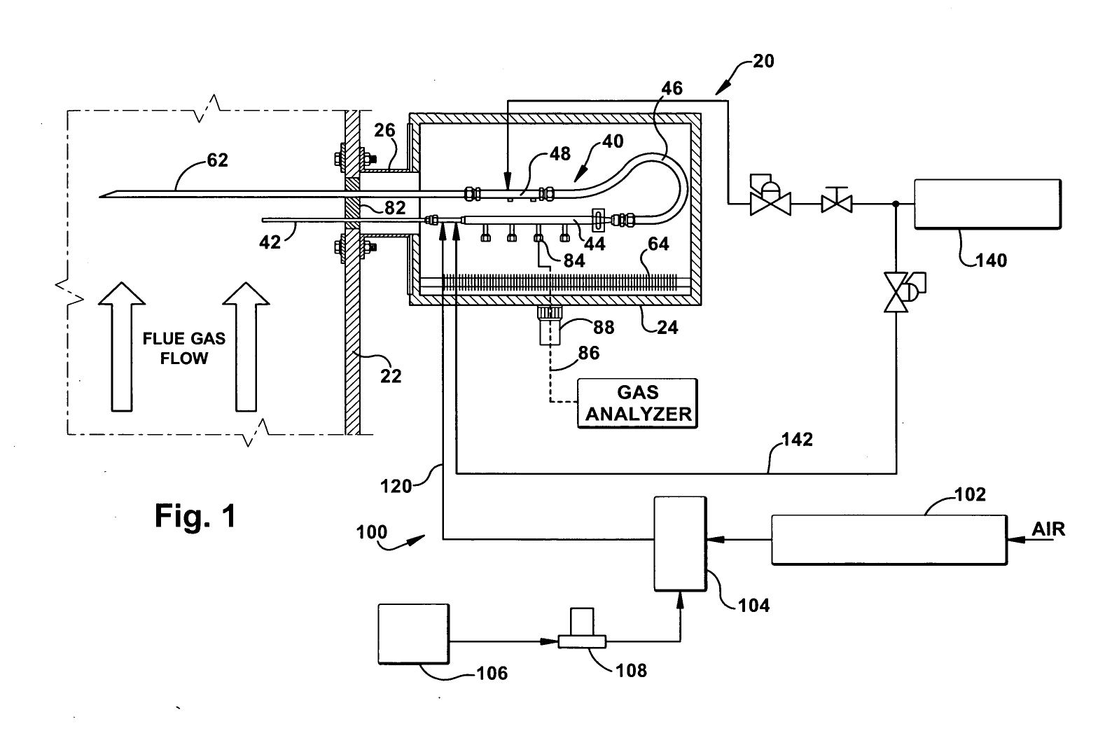

A gas sample acquisition apparatus 20 is illustrated in FIG. 1, and includes structure according to one aspect of the invention for checking the calibration of a continuous emissions monitoring system (CEMS). The gas sample acquisition apparatus 20 is part of the continuous emissions monitoring system and is operatively connected with a known gas analyzer. Such a gas sample acquisition apparatus 20 and CEMS is suitable for sampling desired pollutants, such as mercury, that are transported in a flue gas stream flowing in an exhaust stack 22 from a combustion source.

The gas sample acquisition apparatus 20 includes a housing 24. The housing 24 is made to comply with NEMA standards and is insulated. The housing 24 is attached to the exhaust stack 22 by a tubular connector 26.

The gas sample acquisition apparatus 20 also includes a probe assembly 40 mounted in the housing 24. Components of the probe assembly 40 are tubular. The probe assembly 40 includes an inlet or probe tip 42 that is in fluid communication with the flue gas stream in the exhaust stack 22. The probe tip 42 is connected to an inertial filter 44 of the probe assembly 40. The inertial filter 44 is attached to a generally U-shaped stainless steel return pipe 46. The stainless steel return pipe 46 is attached to a venturi flow meter 48. The venturi flow meter 48 is connected to an outlet or eductor 62 that is open to the flue gas flow. The temperature of the gas sample within the components of the probe assembly 40 located in the housing 24 is maintained via a block or jacket heater 64.

The probe tip 42 extends into the exhaust stack 22 through flexible thermal insulation 82. The probe tip 42 draws a sample from the exhaust flue gas flow. The gas sample is transported into the inertial filter 44. The gas sample leaves the inertial filter 44 via the stainless steel return pipe 46. The gas sample then passes through the venturi flow meter 48. Finally, the gas sample leaves the component housing 24 by passing through the eductor 62. The gas sample is extracted from the gas sample acquisition apparatus 20 via a sample pump (not shown) and a valve (not shown).

During the circulation of the gas sample through the components of the probe assembly 40, a representative sub-sample is drawn from the inertial filter 44 at tap 84. The sub-sample is conducted out of the housing 24 in line 86 extending through port 88. The sub-sample is conducted to a gas analyzer for analysis in a known manner. Suitable gas analyzers are well known in the art and include, without limitation, UV atomic absorption and atomic fluorescence detectors.

It is desirable, but not required, to keep the components of the probe assembly 40 at around 200° C. to ensure optimum accuracy in the measurement of total gaseous mercury concentration. The entire flow path throughout the tubular components of the probe assembly 40 is relatively smooth, with no gaps in the tubing of the assembly where particulate material might collect. Accordingly, the components provide, a consistently laminar flow of the sample through the tubular components of the probe assembly 40 in contact with the flue gas sample. The size and porosity of the inertial filter 44 and other components are selected to provide the desired flow of the gas sample through the components of the probe assembly 40.

The inertial filter 44 is typically made from a tubular sintered metal material. The sintered metal of the inertial filter 44 has a relatively large surface area. The surfaces of the inertial filter 44 act to contact particulates in the exhaust gas which tend to then remove mercury from the exhaust gas by adsorption. Particulates and other undesirable material from the stack gas sample might adhere to the wetted surfaces of the probe and cause the adsorption of elemental mercury onto the wetted surfaces. This adversely affects the concentration of mercury, or analyte, that the gas analyzer is exposed to and is, therefore, not a true measure of the concentration of mercury in the exhaust gas.

To minimize particulate matter from accumulating on surfaces of the components of the probe assembly 40 of the gas sample acquisition apparatus 20 is a calibration checking device 100. The calibration checking device 100 may be mounted to the housing 24 or an external location but is operatively attached to the probe assembly 40. The calibration checking device 100 serves to periodically remove or dislodge the mercury that was removed from the exhaust gas and accumulated on surfaces of the probe assembly 40. Thus, the probe assembly 40 is relatively maintenance free and provides a representative sample from the exhaust flue gas flow to assure the accuracy and precision of the CEMS.

The calibration checking device 100 according to one aspect of the invention includes an elemental mercury sample source 102. The elemental mercury sample source 102 is fluidly connected to a humidifier 104 in the form of a vaporizer. A source of moisture 106 is fluidly connected to the humidifier 104 through a mass flow controller 108. The humidifier is fluidly connected to the probe assembly 40 at the probe tip 42 by a line 120. An air cleanup panel 140 is fluidly connected to the probe tip 42 by line 142.

The calibration checking device 100 provides a humidified sample of a known quantity of elemental mercury to the probe tip 42. The level of humidity is in the range of 2 to 33 percent and preferably in the range of 5-20 percent. It has been found that a humidified sample of elemental mercury provides more accurate and precise measure of mercury than by supplying a dry sample. This is believed due to a cleansing action of the moisture on the particulates and other undesirable material on the wetted surfaces (where the analyte comes into contact) of the probe assembly 40.

The elemental mercury sample source 102 of the calibration checking device 100 provides a flow of a known concentration of elemental mercury to the humidifier 104. The concentration of elemental mercury is, for example 10 micrograms per cubic meter of air (μg/m³). This sample of elemental mercury passes through the vaporizer form of the humidifier 104. A desired amount of moisture is provided from the source 106, such as liquid water, at a temperature above the dew point of the water such as about 70° C. The mass controller 108 provides the desired amount of water. The water is delivered to the flow of elemental mercury sample as moisture vapor. The moisture is carried along with the mercury sample to the probe assembly 40 via line 120. The moisture acts to cleanse the accumulated mercury that was adsorbed onto the surfaces of the probe assembly 40. The moisture acts to cleanse particulates and other undesirable material that are adhering to the wetted surfaces of the probe and eliminates the adsorption of elemental mercury from the stack gas sample or the calibrated elemental mercury gas and thereby provide an accurate measure of the concentration of the gaseous species of mercury. Thus, the sample of elemental mercury that the gas analyzer measures is representative of the concentration delivered by the source 102.

The purpose of this aspect of the invention is to not enhance the elemental mercury calibration gas but to provide a “cleansing” solution along with the elemental mercury calibration gas to wash away any particulates and other undesirable material that cause the adsorption of elemental mercury from the stack gas sample or the elemental mercury calibration gas onto the wetted surfaces of the probe where the analyte comes into contact with. The removal of elemental Hg from the sample gas, whether it is stack gas sample or calibration sample, affects the accuracy and precision of the measurement of the elemental Hg. By preventing this removal a more accurate and precise measurement of the analyte is made for the stack gas sample and calibration gas.

To insure that the gas analyzer provides the most precise and accurate measurement of the analyte, a calibration checking system 100. The calibration checking system 100 is in fluid communication with the probe. The calibration checking system includes a source that provides a known concentration of calibration material to be measured by the analyzer. The calibration material is the same as the analyte. A humidifier is associated with the source to provide moisture to a flow of calibration material. The moisture acts to cleanse particulates and other undesirable material from the probe and thereby provide an accurate measure of the concentration of the calibration material.

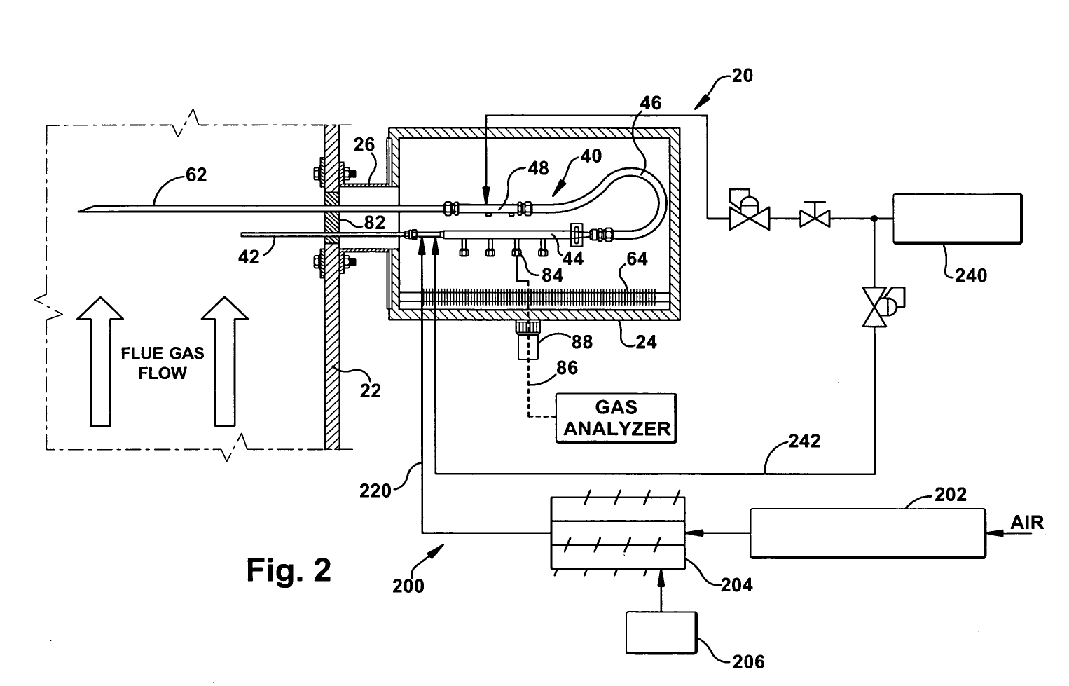

A gas sample acquisition apparatus 20 is illustrated in FIG. 2, and includes structure according to another aspect of the invention for checking the calibration of a continuous emissions monitoring system (CEMS). The gas sample acquisition apparatus 20 is part of the continuous emissions monitoring system and is operatively connected with a known gas analyzer. Such a gas sample acquisition apparatus 20 and CEMS is suitable for sampling desired pollutants, such as mercury, that are transported in a flue gas stream flowing in an exhaust stack 22.

The gas sample acquisition apparatus 20 includes the housing 24. The housing 24 is attached to the exhaust stack 22 by the tubular connector 26. The probe assembly 40 includes the probe tip 42 connected to the inertial filter 44. The inertial filter 44 is attached to the return pipe 46. The return pipe 46 is attached to the venturi flow meter 48. The venturi flow meter 48 is connected to the eductor 62 that is open to the flue gas flow. The temperature of the components of the gas sample acquisition apparatus 20 is maintained via a block or jacket heater 64.

To minimize particulate matter from accumulating on surfaces of the components of the probe assembly 40 of the gas sample acquisition apparatus 20 is a calibration checking device 200. The calibration checking device 200 may be mounted to the housing 24 or an external location but is operatively attached to the component of the probe assembly 40. The calibration checking device 200 serves to periodically remove or dislodge the mercury that was removed from the exhaust gas and accumulated on surfaces of the probe assembly 40. Thus, the probe assembly 40 is relatively maintenance free and provides a representative sample from the exhaust flue gas flow to assure the accuracy and precision of the CEMS.

The calibration checking device 200 according to one aspect of the invention includes an elemental mercury sample source 202. The elemental mercury sample source 102 is fluidly connected to a humidifier 204 in the form of a permeation tube. A source of moisture 206 is fluidly connected to the humidifier 204. The humidifier 204 is fluidly connected to the probe assembly 40 at the probe tip 42 by a line 220. An air cleanup panel 240 is fluidly connected to the probe tip 42 by line 242.

The calibration checking device 200 provides a humidified sample of a known quantity of elemental mercury to the probe tip 42. The level of humidity is in the range of 2 to 33 percent and preferably in the range of 5-20 percent. It has been found that a humidified sample of elemental mercury provides more accurate and precise measure of mercury than by supplying a dry sample. This is believed due to a cleansing action of the moisture on the particulates and other undesirable material accumulated on the surfaces of the probe assembly 40.

The elemental mercury sample source 202 of the calibration checking device 200 provides a flow of a known concentration of elemental mercury to the humidifier 204. The concentration of elemental mercury is, for example 10 micrograms per cubic meter of air (μg/m³). This sample of elemental mercury passes through the permeation tube from of the humidifier 204. A desired amount of moisture is provided from the source 206, such as liquid water. The water is delivered to the flow of elemental mercury sample as moisture vapor. The moisture is carried along with the mercury sample to the probe assembly 40 via line 220. The moisture acts to cleanse the accumulated mercury that was adsorbed onto the surfaces of the probe assembly 40. Thus, the sample of elemental mercury that the gas analyzer measures is representative of the concentration delivered by the source 202.

To insure that the gas analyzer provides the most precise and accurate measurement of the analyte, a calibration checking system 200. The calibration checking system 200 is in fluid communication with the probe. The calibration checking system includes a source that provides a known concentration of calibration material to be measured by the analyzer. The calibration material is the same as the analyte. A humidifier is associated with the source to provide moisture to a flow of calibration material. The moisture acts to cleanse particulates and other undesirable material from the probe that could cause the adsorption of elemental Hg onto the wetted surfaces of the probe and thereby provide an accurate measure of the concentration of the calibration material.

Calibration error checks with calibration gas humidified by the permeation tube version of the humidifier 204 provided good recovery and relatively fast responses. “Zero gas” check responses were within the “pass” window by the second reading. “Span gas” check responses were within the ±1 μg/m³ “pass” window by the third reading.

Results of the calibration error checks are summarized in the table below.

| | Zero Gas | | Span Gas | |
|---|---|---|---|---|
| | ±1 μg/m³ | | 9.1 ± 1 μg/m³ | |
| Test Number | reading | 10^{th} reading | reading | 10^{th} reading |
| 1 | 2^{nd} reading | −0.1 μg/m³ | 3^{rd} reading | 9.1 μg/m³ |
| | (0.2 μg/m³) | | (8.6 μg/m³) | |
| 2 | 2^{nd} reading | −0.2 μg/m³ | 3^{rd} reading | 9.2 μg/m³ |
| | (0.1 μg/m³) | | (8.7 μg/m³) | |
| 3 | 2^{nd} reading | −0.3 μg/m³ | 3^{rd} reading | 9.1 μg/m³ |
| | (0.2 μg/m³) | | (8.6 μg/m³) | |

While the invention has been described in terms of various specific embodiments, those skilled in the art will recognize that the invention can be practiced with modification within the spirit and scope of the claims.

What is claimed is:

1. A continuous emissions monitoring system that is in fluid communication with a flue stack conducting exhaust gas from a combustion source, the continuous emissions monitor system comprising:
an analyzer for measuring concentrations of an analyte present in the exhaust gas;
a probe in fluid communication with the flue stack to acquire a sample of exhaust gas from the flue stack and in fluid communication with and located upstream of the analyzer, the probe tending to remove analyte from the sample; and
a calibration checking system in fluid communication with the probe, the calibration checking system including:
a source that provides a flow of a known concentration of calibration material to be measured by the analyzer, the calibration material being chemically the same as the analyte; and
a humidifier associated with the source that humidifies the flow of calibration material, the moisture acting to cleanse removed analyte from the probe and thereby enable an accurate measurement of the concentration of the calibration material.

2. The continuous emissions monitoring system of claim 1 wherein the humidifier includes a vaporizer.

3. The continuous emissions monitoring system of claim 1 wherein the humidifier includes a permeation tube.

4. The continuous emissions monitoring system of claim 1 wherein the analyte is mercury.

5. The continuous emissions monitoring system of claim 1 wherein the source provides elemental mercury in a gaseous state.

6. The continuous emissions monitoring system of claim 1 wherein the analyzer measures the concentration of calibration material provided by the source with an accuracy of plus or minus ten percent.

7. An improved continuous emissions monitoring system that is in fluid communication with a flue stack conducting exhaust gas from a combustion source, the continuous emissions monitor system having an analyzer for measuring concentrations of mercury present in the exhaust gas; a probe in fluid communication with the flue stack to acquire a sample of exhaust gas from the flue stack and in fluid communication with and located upstream of the analyzer, the probe tending to remove mercury from the sample; and a calibration checking system in fluid communication with the probe, the calibration checking system including a source that provides a flow of a known concentration of a gaseous species of mercury to be measured by the analyzer; and wherein the improvement comprises:
a humidifier operatively connected with the source to provide moisture to gaseous species of mercury flowing through the humidifier, the moisture acting to cleanse removed mercury from the probe and thereby enable accurate measurement of the concentration of the gaseous species of mercury.

8. The improved continuous emissions monitoring system of claim 7 wherein the humidifier includes a vaporizer.

9. The improved continuous emissions monitoring system of claim 7 wherein the humidifier includes a permeation tube.

10. A method of continuous emissions monitoring of a flue stack conducting exhaust gas from a combustion source, the method comprising the steps of:
acquiring a sample of exhaust gas from the flue stack with a probe, the probe tending to remove analyte from the sample;
measuring concentrations of the analyte with an analyzer located downstream of the probe; and
checking the calibration of the analyzer with a flow of a known concentration of calibration material provided by a source, the calibration material being chemically the same as the analyte; and
humidifying the flow of calibration material with moisture, the moisture acting to cleanse removed analyte from the probe and thereby enable an accurate measurement of the concentration of the calibration material.

11. The method of continuous emissions monitoring of claim 10 wherein the flow of calibration material is humidified with a vaporizer.

12. The method of continuous emissions monitoring of claim 10 wherein the flow of calibration material is humidified with a permeation tube.

13. The method of continuous emissions monitoring of claim 10 wherein the removed analyte is mercury.

14. The method of continuous emissions monitoring of claim 10 wherein the calibration material is elemental mercury in a gaseous state.
